(19) United States
(12) Patent Application Publication
(10) Pub. No.: US 2008/0171983 A1
(43) Pub. Date: Jul. 17, 2008

(54) Needle hub assembly

(76) Inventor: Eric J. Knutson, Granite Bay, CA (US)

Correspondence Address: Eric J. Knutson, 5815 Granite Hills Drive, Granite Bay, CA, 95746, US

(21) Appl. No.: 11/588,917

(22) Filed: Oct. 27, 2006

(51) Int. Cl. A61M 5/00

(52) U.S. Cl. 604/117

(57) ABSTRACT

A needle-hub assembly, the improvement comprising a small tissue deflector connected to the needle, wherein the deflector deflects tissues away from the hub to prevent needle penetration to the hub, but permits the needle to bend adjacent to the hub, thereby enhancing the retrievability of the needle from the tissues if the needle were to separate.

### BACKGROUND

1. Field of Invention

This invention relates to healthcare and veterinary care, specifically to needles.

2. Description of Prior Art

Needle breakage remains a problem in healthcare and veterinary care. Although greatly reduced by the advent of disposable needles, needle breakages still occur, and generally result in a minor surgical procedure being required to extricate the needle fragment.

Needles tend to break near the hub. Most often, they break because the needle is bent during use, both intentionally and unintentionally. The problem with broken needles occurs when the needle is inserted fully and the hub is pushing against the tissues. When a fully inserted needle breaks off, the tissues tend to close over the insertion point hole as the hub is withdrawn, thereby covering the needle fragment. Non-surgical attempts to retrieve the fragment generally result in pushing it deeper into the tissues .

To accomplish surgical recovery of the needle fragment, the patient must be informed, and anesthetized. In humans, broken needle incidents tend to result in litigation. In animals, anesthesia and surgery is costly, or worse, sometimes the broken needles are simply left in the animal, which causes a detection problem for the meat industry.

Most broken needle incidents could be prevented by avoiding full insertion of the needle to the hub. Needle lengths selected by manufacturers typically includes several millimeters of additional length beyond the usual anticipated depth of needle penetration. The additional length is provided to reduce the need to insert needles to the hub. However, in many cases, practitioners find that allowing the hub to stop the needle insertion is more efficient. In some cases, full insertion of the needle is also more effective.

Avoiding needle bends could also reduce needle breakage, but, again, practitioners often find that bending a needle can increase effectiveness, especially when working in a restricted space.

A few safety syringes include a needle depth restrictor that is attached to the syringe barrel, and may be extended down from the barrel to prevent needle insertion to the hub. The depth restrictors tend to be large. Typically the width is similar to the barrel. The large size of the restrictors can cause interference with routine use, especially when used in limited access sites. In some types, the user has the option to disable the depth restrictor, or the option not to engage it. In addition, the restrictor may limit how the needle can be bent, and thereby interferes with efficient and effective access of needle use.

The above needle depth restrictors suffer from a number of disadvantages:
(a) Users can opt not to use restrictors
(b) Restrictors are large, and interfere with access
(c) Restrictors interfere with needle bending to optimize angulation
(d) Restrictors are costly

A device similar to my needle hub assembly with novel tissue deflector has not been considered by many medical inventors because most do not know that needle breakage remains a significant problem. The few inventors who are aware of the problem are largely disinterested in depth restrictors due to their lack of popularity.

### OBJECTS AND ADVANTAGES

Accordingly, several objects and advantages of my needle hub assembly with a novel tissue deflector are:
(a) the deflector is functional as manufactured, and is difficult to disable
(b) The deflector is sufficiently small so as to not interfere with limited access sites
(c) The deflector does not substantially interfere with needle bending
(d) The deflector does not add substantially to the cost of a needle-hub assembly

Further objects and advantages are to provide a needle hub assembly that does not alter user technique, and does not distract the user, in order to prevent needle breakage.

Still further objects and advantages will become apparent from a consideration of the ensuing description and drawings.

### DRAWING FIGURES

In the drawings, closely related figures have the same number, but different alphabetic suffixes.

FIG. 1 is a cross-sectional view showing a sleeve-shaped deflector connected to the portion of a needle that is proximal to the hub, where the deflector connects directly to the hub.

FIG. 2 is a perspective view showing a sleeve-shaped deflector connected to the portion of a needle that is proximal to the hub, where the deflector connects directly to the hub. The deflector is shown with a meshwork of multiple perforations adjacent to the hub, to enhance flexibility of the deflector during needle bending, and with a needle bevel indicator.

FIG. 3 is a perspective view showing a sleeve-shaped deflector connected to the portion of a needle that is proximal to the hub, where the deflector connects to the hub. The deflector is shown with a needle bevel indicator, with perforations adjacent to the hub to enhance flexibility of the deflector during needle bending, and with rings at intervals along the deflector, to ensure tissue deflection.

FIG. 4A-4C are perspective views showing various embodiments of deflectors connected to the needle proximal to the hub, where the deflector does not connect to the hub.

FIG. 5 is a perspective view showing a deflector connected to the hub with a connector.

### REFERENCE NUMERALS In DRAWINGS

| | | | |
|---|---|---|---|
| 10 | deflector | 12 | needle |
| 14 | hub | 16 | stop |
| 18 | indicator | 20 | perforation |
| 22 | ring | 24 | connector |

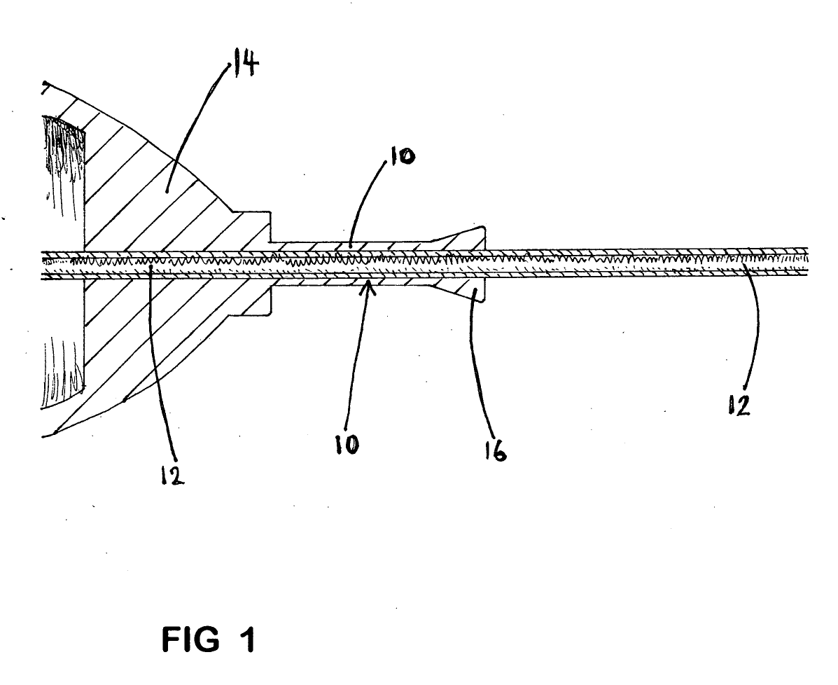

### Description—FIGS. 1 to 5

The invention provides an improved needle hub assembly, a typical embodiment of which is shown in FIG. 1. The needle hub assembly comprises a tissue deflector, deflector 10, connected to a needle, needle 12, and needle 12 is connected to a hub, hub 14, wherein deflector 10 is substantially larger than the diameter of needle 12, such that deflector 10 is of sufficient size to deflect tissue away from hub 14 when needle 12 is inserted into tissues. Deflector 10 is shown connected directly to hub 14. A raised end, stop 16, is shown at the distal end of deflector 10, stop 16 having a greater diameter than the remainder of deflector 10 to further facilitate tissue deflection.

It is preferred that deflector 10 permits a bend to be formed along the length of needle 12, said bend being located between hub 14 and at least a portion of deflector 10.

It is preferred that at least a portion of deflector 10 is flexible, so as to permit bending of needle 12 adjacent to hub 14. However, deflector 10 may be substantially non-flexible, such that deflector 10 fractures to permit needle bending adjacent to hub 14, or such that deflector 10 inhibits needle bending.

It is preferred that deflector 10 is non-uniform in diameter over the length of deflector 10, such that the diameter of deflector 10 is sufficiently wide at the end distal from hub 14 to deflect the tissues away from hub 14, and that the diameter of deflector 10 is minimized at the end proximal to hub 14 to be flexible and facilitate bending of needle 12 at that location. However, the diameter of deflector 10 may be uniform.

It is preferred that deflector 10 is comprised of a material that is identical to the material of which hub 14 is comprised, such as plastic, metal, and so on. It is preferred that deflector 10 comprises an extension of hub 14 over needle 12. However, deflector 10 material may be comprised of a material that is different than hub 14 material. Other materials which may comprise deflector 10 include solder, glue, ceramic, a widened portion of needle 12, a very thick layer of paint, and so on.

It is preferred that deflector 10 extend along needle 12 in the range of 2-10 mm from hub 14, with an extension of about 5 mm considered ideal.

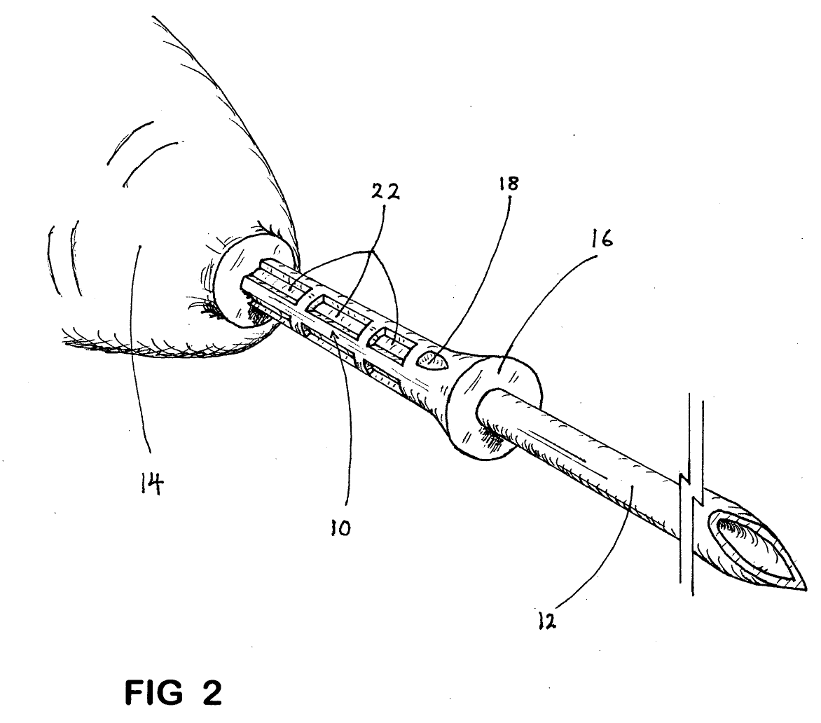

FIG. 2 shows a sleeve-shaped deflector 10 connected to the portion of needle 12 that is proximal to hub 14, where deflector 10 connects directly to hub 14. Deflector 10 is shown as an extension of hub 14, wherein deflector 10 is comprised of the same material as hub 14. Deflector 10 has multiple perforations 20 forming a meshwork adjacent to hub 14, thereby enhancing the flexibility of deflector 10 during bending of needle 12. A needle bevel indicator 18 is also shown.

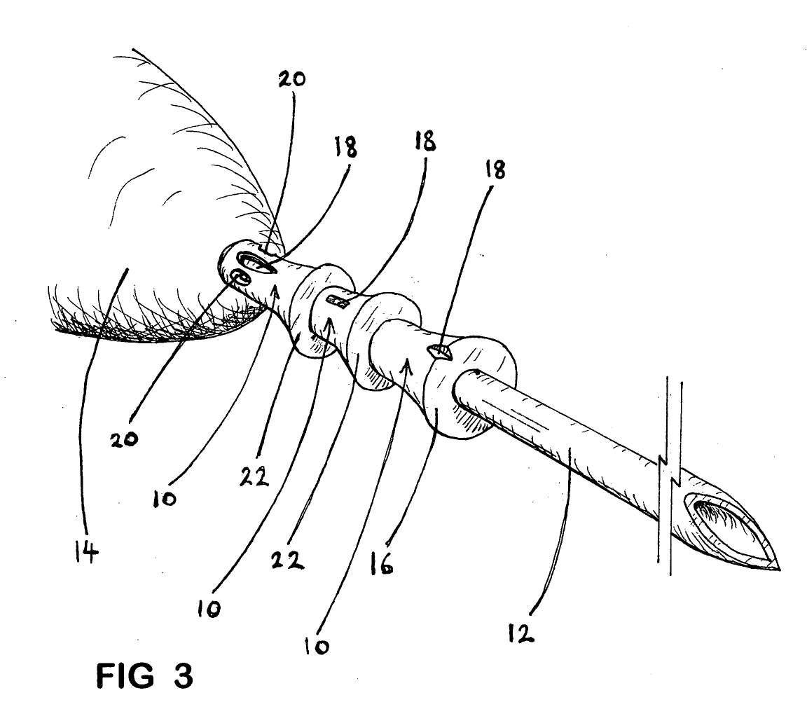

FIG. 3 shows a sleeve-shaped deflector 10 connected to the portion of needle 12 that is proximal to hub 14, and deflector 10 is connected directly to hub 14. Needle 12 is shown with a bend proximal to hub 14. An indicator, indicator 18, is aligned with the bevel of needle 12 to facilitate bending of needle 12. Deflector 10 is shown having perforations, perforation 20, in the portion of deflector 10 that is proximal to hub 14, so as to enhance the flexibility of deflector 10, and thereby facilitate bending of needle 12 proximal to hub 14.

Also shown are a multiplicity of rings, ring 22, spaced at intervals along deflector 10, wherein rings 22 have a greater diameter than the remainder of deflector 10, such that rings 22 further facilitate the tissue deflection function of deflector 10.

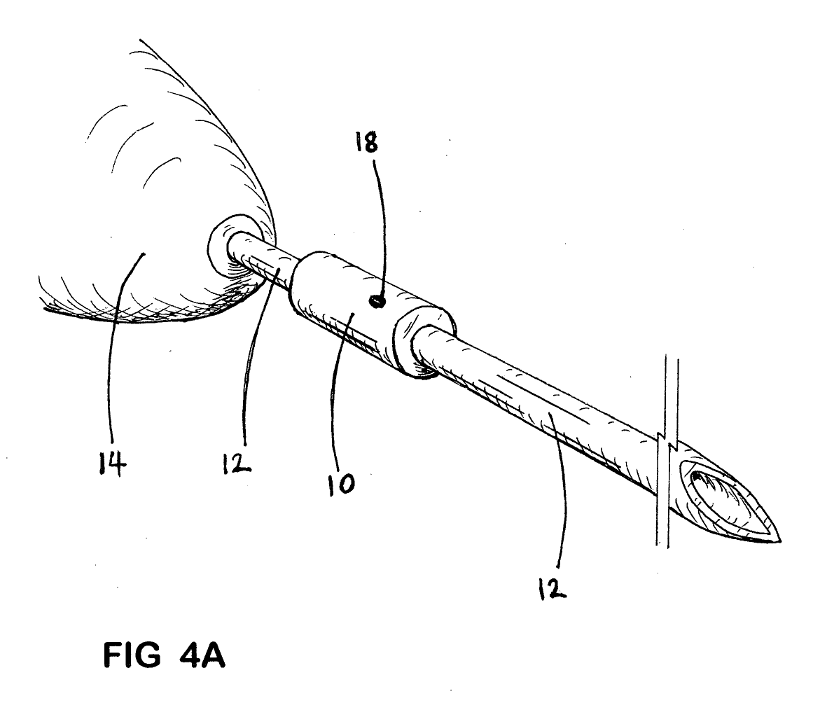

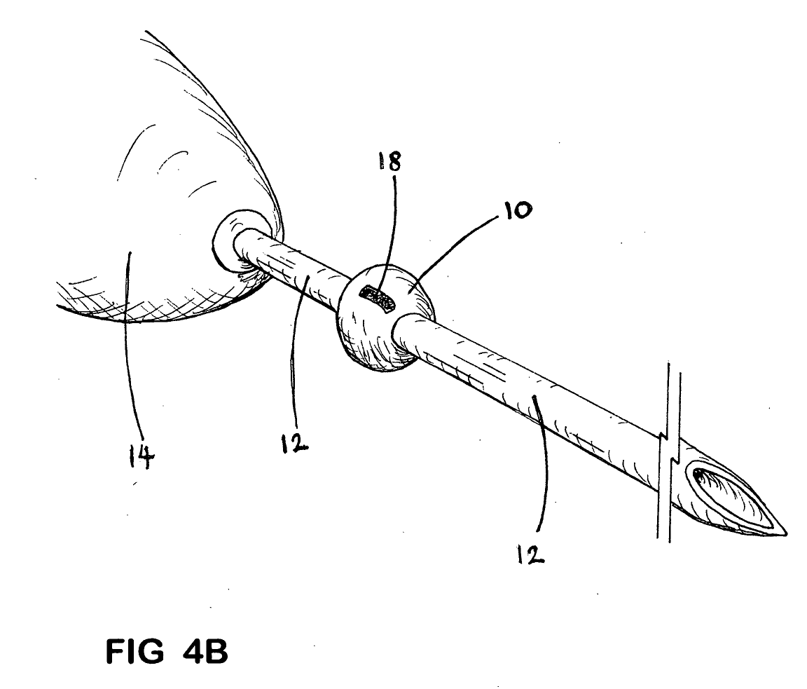

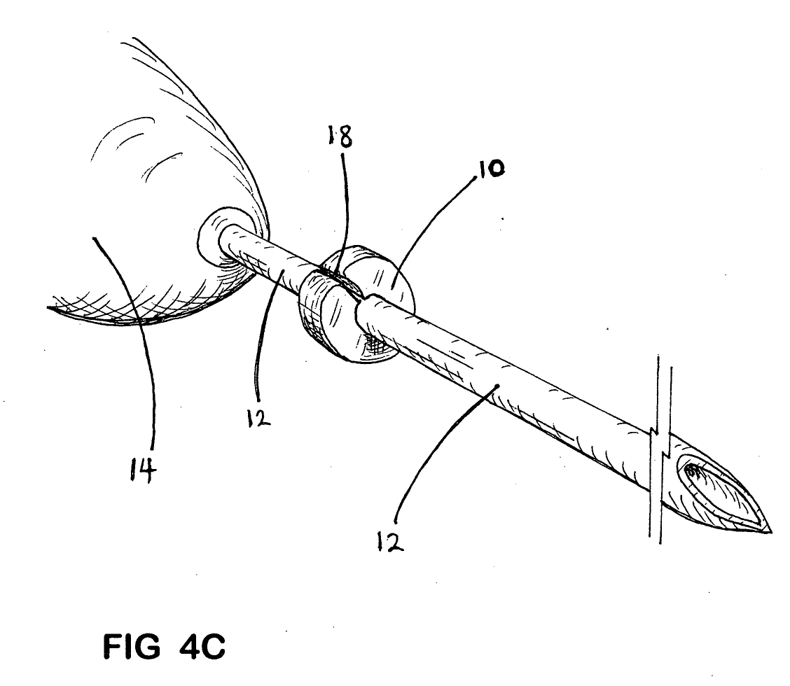

Additional embodiments of deflector 10 are shown in FIGS. 4A-4C, where deflector 10 is connected to needle 12 but is not connected directly to hub 14, and therefore a portion of needle 12 is interposed between deflector 10 and hub 14. Deflector 10 is shown with an indicator 18 aligned with the bevel of needle 12.

FIG. 4A shows a sleeve-shaped deflector 10. It is preferred that said sleeve-shaped deflector 10 is non-slidingly connected to needle 12. However, said sleeve-shaped deflector 10 may be slidingly connected to needle 12, such that the location of said sleeve-shaped deflector 10 on needle 12 may be changed, or said sleeve-shaped deflector 10 may be introduced onto needle 12 by user.

FIG. 4B shows a node-shaped deflector 10, wherein deflector 10 is comprised of a small mass of plastic, glue, solder, adhered to needle 12, or an expanded portion of needle 12, and so on.

FIG. 4C shows a clamp-type deflector 10, wherein a deflector 10 comprised of metal, plastic, or other materials, connects to needle 12 with pressure and friction.

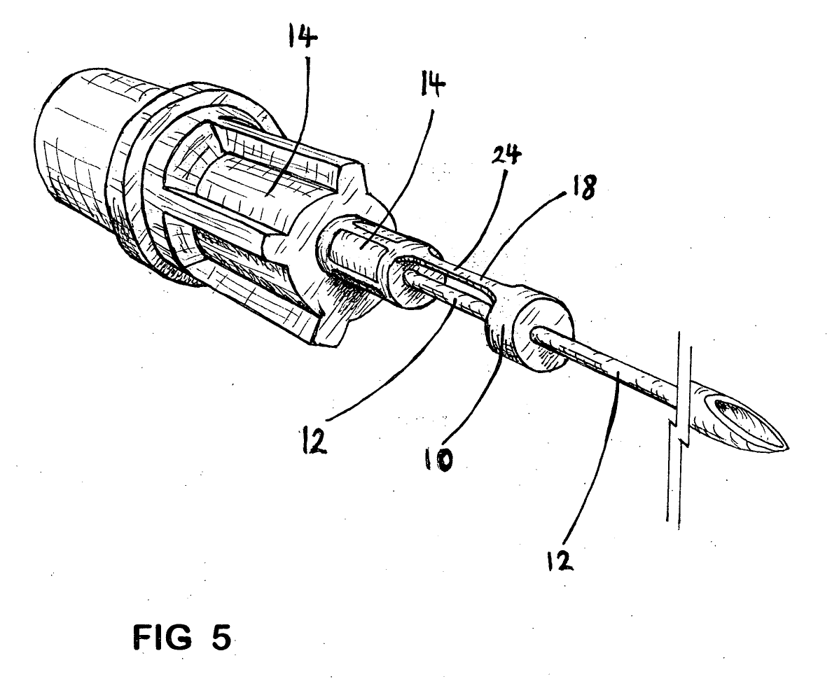

FIG. 5 shows a connector, connector 24, connecting deflector 10 to hub 14.

It is preferred that connector 24 has a specified orientation relative to the bevel of needle 12, to facilitate bending of needle 12, so that connector 24 functions as indicator 18.

It is preferred that connector 24 is sufficiently flexible to permit a bend to be formed along the length of needle 12, said bend being located between hub 14 and at least a portion of deflector 10. However, connector 24 may be inflexible so as to prevent bending needle 12 proximal to hub 14, or connector 24 may fracture to permit bending needle 12 proximal to hub 14.

From the description above, a number of advantages of the needle hub assembly with deflector become evident:
(a) the deflector is functional as manufactured
(b) the deflector does not interfere with access
(c) the deflector permits needle bending
(d) the deflector is economical

### Operation—FIGS. 1-5

By using the needle hub assembly of the invention, it is now possible, surprisingly, to retrieve a separated needle despite full insertion up to deflector 10.

The needle hub assembly offers the advantage that the user can now give injections or withdraw samples without burdensome needle-breakage precautions, and with confidence that a separated needle would be retrievable.

### EXAMPLE 1

User selects a needle hub assembly having a flexible deflector 10 which is connected directly to hub 14, as shown in FIGS. 1, 2, and 3. User refers to indicator 18 to identify the angle of the needle bevel, and decides the angulation of the needle bend. User applies lateral pressure against needle 12, and flexible deflector 10 flexes to permit needle 12 to bend proximal to hub 14.

User forcefully inserts the full distal portion of needle 12 into the tissues until stop 16 portion of deflector 10 pushes against the tissues, thereby displacing the tissues away from the advancing hub 14 until the insertion of needle 12 is halted. Deflector 10 protrudes from the tissues, and the proximal portion of needle 12 and hub 14 are held away from the insertion point of the distal portion of needle 12 into the tissues.

By chance, needle 12 separates adjacent to hub 14 at the location of the bend. Deflector 10 remains connected to the portion of needle 12 that is distal from the point of separation. Deflector 10 also remains connected to hub 14.

When user withdraws hub 14 away from the tissues, deflector 10 withdraws connected needle 12 entirely from the tissues. User may not even become aware that needle 12 has separated from hub 14.

### EXAMPLE 2

User selects a standard needle hub assembly having no deflector 10. User selects a separate sleeve-shaped deflector 10 having a central lumen for receiving a needle. User inserts needle 12 into the lumen of deflector 10, and slides deflector 10 along needle 12 until it is in close proximity to hub 14, as shown in FIG. 4A. Deflector 10 is then connected to needle 12 by friction, but deflector 10 is not connected to hub 14. User applies lateral pressure against needle 12, and deflector 10 permits needle 12 to bend proximal to hub 14.

User forcefully inserts the full distal portion of needle 12 into the tissues until stop 16 portion of deflector 10 pushes against the tissues, thereby displacing the tissues away from the advancing hub 14 until the insertion of needle 12 is halted. Deflector 10 protrudes from the tissues, and the proximal portion of needle 12 and hub 14 are held away from the insertion point of the distal portion of needle 12 into the tissues.

By chance, needle 12 separates from hub 14 proximally to hub 14. Deflector 10 remains connected to needle 12 distal from the point of separation.

When user withdraws hub 14 away from the tissues, hub 14 separates from deflector 10 and broken needle 12, but deflector 10 remains connected to needle 12. As the pressure exerted on the tissues by hub 14 is withdrawn, the tissues rebound, and push against deflector 10, and thereby move deflector 10 with still-connected needle 12 outward a few millimeters until the tissues are completely rebounded. Deflector 10 and a portion of needle 12 protrude above the surface of the tissues. After user becomes aware that needle 12 has separated from hub 14, user grasps deflector 10 and withdraws deflector 10 with connected needle 12 from the tissues.

### EXAMPLE 3

User connects a needle hub assembly having a deflector 10 which is not connected to hub 14, as shown in FIGS. 4B and 4C. User applies lateral pressure against needle 12, and deflector 10 permits needle 12 to bend proximal to hub 14.

User forcefully inserts the full distal portion of needle 12 into the tissues until deflector 10 pushes against the tissues, thereby displacing the tissues away from the advancing hub 14 until the insertion of needle 12 is halted. Deflector 10 protrudes from the tissues, and the proximal portion of needle 12 and hub 14 are held away from the insertion point of the distal portion of needle 12 into the tissues.

By chance, needle 12 separates from hub 14 proximally to hub 14. Deflector 10 remains connected to needle 12 distal from the point of separation.

When user withdraws hub 14 away from the tissues, hub 14 separates from deflector 10 and broken needle 12, but deflector 10 remains connected to needle 12. As the pressure exerted on the tissues by hub 14 is withdrawn, the tissues rebound, and push against deflector 10, and thereby move deflector 10 with still-connected needle 12 outward a few millimeters until the tissues are completely rebounded. Deflector 10 and a portion of needle 12 protrude above the surface of the tissues. After user becomes aware that needle 12 has separated from hub 14, user grasps deflector 10 and withdraws deflector 10 with connected needle 12 from the tissues.

### EXAMPLE 4

User connects a needle hub assembly having a connector 24 connected to hub 14, as shown in FIG. 5. For this example, connector 24 is brittle and inflexible. User applies pressure against needle 12 in an attempt to bend needle 12. The pressure fractures brittle connector 24, so that deflector 10 becomes disconnected from hub 14, but remains connected to needle 12. The pressure bends needle 12 proximal to hub 14.

User forcefully inserts the full distal portion of needle 12 into the tissues until stop 16 portion of deflector 10 pushes against the tissues, thereby displacing the tissues away from the advancing hub 14 until the insertion of needle 12 is halted. Deflector 10 protrudes from the tissues, and the proximal portion of needle 12 and hub 14 are held away from the insertion point of the distal portion of needle 12 into the tissues.

By chance, needle 12 separates from hub 14 proximally to hub 14. Deflector 10 remains connected to needle 12 distal from the point of separation.

When user withdraws hub 14 away from the tissues, hub 14 separates from deflector 10 and broken needle 12, but deflector 10 remains connected to needle 12. As the pressure exerted on the tissues by hub 14 is withdrawn, the tissues rebound, and push against deflector 10, and thereby move deflector 10 with still-connected needle 12 outward a few millimeters until the tissues are completely rebounded. Deflector 10 and a portion of needle 12 protrude above the surface of the tissues. After user becomes aware that needle 12 has separated from hub 14, user grasps deflector 10 and withdraws deflector 10 with connected needle 12 from the tissues.

### SUMMARY, RAMIFICATIONS AND SCOPE

Accordingly, the reader will see that the needle hub assembly of this invention could prevent the complications from most broken-needle incidents. The deflector can facilitate retrieving most separated needles by initially preventing insertion of the entire needle length, and secondarily, by maintaining a connection to the fractured needle fragment. Furthermore, the needle hub assembly has the additional advantages in that it facilitates retrievability of separated needles very economically.

The presence of deflector 10 with a needle hub assembly should not necessitate manufacturing needles 12 with increased length to accommodate deflector 10. Manufacturers typically provide an additional length of several millimeters beyond the usual depth of needle penetration. Deflector 10 would connect to needle 12 substantially within that additional length all ready provided for most needles.

Although the description above contains many specificities, these should not be construed as limiting the scope of the invention and process, but as merely providing illustrations of some of the presently preferred embodiments of this invention.

For example, deflector 10 may be comprised of a portion of a needle cap or sheath, such that a deflector 10 portion of the needle cap remains connected to needle 12 after the remaining portion of the needle cap is removed from needle 12 prior to use.

For another example, hub 14 may be continuous and integral with a syringe, so that needle 12 is connected directly to a hub area of a syringe, rather than to a separate hub 14.

Thus the scope of the invention should be determined by the appended claims and their legal equivalents, rather than by the examples given.

I claim:

1. A needle-hub assembly comprising a needle connected to a hub, the improvement comprising a tissue deflector connected to said needle, wherein said deflector is shaped to inhibit insertion of said deflector into the tissues, thereby enhancing the retrievability of a separated said needle from the tissues.

2. The needle-hub assembly of claim 1, wherein said deflector is connected directly to said hub.

3. The needle-hub assembly of claim 1, wherein said deflector is not connected to said hub.

4. The needle-hub assembly of claim 4, wherein said deflector is substantially comprised of a small mass adhered to said needle.

5. The needle-hub assembly of claim 1, wherein said deflector is substantially comprised of a sleeve.

6. The needle-hub assembly of claim 1, wherein said deflector indicates the location of the bevel of said needle.

7. A needle-hub assembly comprising a needle connected to a hub, the improvement comprising a tissue deflector connected to said needle, said deflector having an end distal from said hub called a stop, and said needle having a portion interposed between said stop and said hub called a proximal needle, wherein said deflector permits user to bend said proximal needle, and wherein said deflector is shaped to inhibit insertion of said deflector into the tissues, thereby enhancing the retrievability of a separated said needle from the tissues.

8. The needle-hub assembly of claim 7, wherein said deflector is connected directly to said hub.

9. The needle-hub assembly of claim 7, wherein said deflector is not connected to said hub.

10. The needle-hub assembly of claim 9, wherein said deflector is comprised of a small mass adhered to said needle.

11. The needle-hub assembly of claim 7, wherein said deflector is substantially comprised of a sleeve.

12. The needle-hub assembly of claim 7, wherein said deflector permits bending of said needle between said deflector and said hub.

13. The needle-hub assembly of claim 7, wherein said deflector indicates the location of the bevel of said needle.

14. A needle-hub assembly comprising a needle connected to a hub, the improvement comprising a tissue deflector connected to a location along the length of said needle, wherein said deflector is shaped to inhibit insertion of said deflector into the tissues, such that said deflector permits said needle to penetrate to only to the location of said deflector, and inhibits needle penetration beyond the location of said deflector, thereby enhancing the retrievability of a separated said needle from the tissues.

15. The needle-hub assembly of claim 14, wherein said deflector is connected directly to said hub.

16. The needle-hub assembly of claim 14, wherein said deflector is not connected to said hub.

17. The needle-hub assembly of claim 16, wherein said deflector is comprised of a small mass adhered to said needle.

18. The needle-hub assembly of claim 14, wherein said deflector is substantially comprised of a sleeve.

19. The needle-hub assembly of claim 14, wherein said deflector permits bending of said needle between said deflector and said hub.

20. The needle-hub assembly of claim 14, wherein said deflector indicates the location of the bevel of said needle.
